(19) United States
(12) Patent Application Publication
(10) Pub. No.: US 2007/0135398 A1
(43) Pub. Date: Jun. 14, 2007

(54) Process for the preparation of ciclesonide

(76) Inventors: Pierluigi Rossetto, Balerna (CH); Peter Lindsay MacDonald, Gentilino (CH)

Correspondence Address: KENYON & KENYON LLP, ONE BROADWAY, NEW YORK, NY, 10004, US

(21) Appl. No.: 11/592,475

(22) Filed: Nov. 2, 2006

Related U.S. Application Data
(60) Provisional application No. 60/733,007, filed on Nov. 2, 2005.
(60) Provisional application No. 60/757,789, filed on Jan. 9, 2006.
(60) Provisional application No. 60/779,751, filed on Mar. 6, 2006.

(51) Int. Cl. C07J 71/00; A61K 31/58

(52) U.S. Cl. 514/174; 540/61

(57) ABSTRACT

Provided is a process for increasing the 22R/22S epidemic ratio of ciclesonide.

### RELATED APPLICATIONS

This application claims the benefit of U.S. provisional application Ser. Nos. 60/733,007, filed Nov. 2, 2005; 60/757,789, filed Jan. 9, 2006; and 60/799,751, filed Mar. 6, 2006, hereby incorporated by reference.

### FIELD OF THE INVENTION

The invention encompasses processes for increasing the proportion of the 22-R epimer of ciclesonide in epidemic mixtures of ciclesonide.

### BACKGROUND OF THE INVENTION

Inhaled synthetic glucocorticosteroids are widely used in the therapy of bronchial asthma for which they are the most effective agents available. Regular treatment with inhaled glucocorticoids improves asthma control and lung function, and reduces asthma attacks. This improvement in asthma control is associated with attenuation of markers or airway inflammation, such as airway responsiveness to provocative stimuli, sputum eosiniphilia, and exhaled nitric oxide concentration.

Ciclesonide, pregna-1,4-diene-3,20-dione, 16,17-[[(R)-cyclohexylmethylene]bis(oxy)]-11-hydroxy-21-(2-methyl-1-oxopropoxy)-(11β,16α)-(9CI), has the chemical formula C₃₂H₄₄O₇, molecular weight of 540.69, and the chemical structure: 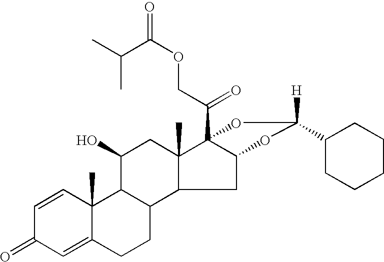
Ciclesonide has 22R configuration.

Ciclesonide is a non-halogenated glucocorticoid with high local anti-inflammatory properties that is inhaled in the treatment of asthma. Ciclesonide is an ester prodrug, essentially devoid of oral bioavailability, which is activated upon cleavage by endogenous esterases (Current Opinion in Investigational Drugs 2002, 3(1) 78-83).

U.S. Pat. No. 5,482,934 discloses the preparation of 16,17-acetals, such as ciclesonide and its 21-hydroxy analogue, from either their corresponding 16α,17-esters by a single-pot deacylation/acetonization sequence employing HCl/dioxane and an additional acid catalyst (toluenesulfonic or perchloric acid) in the presence of a suitable donor, (ie cyclohexane carbaldehyde).

U.S. Pat. Nos. 2,990,401 and 3,929,768 disclose general processes for the preparation of 16,17-acetals, such as ciclesonide, from their corresponding 16α,17-diols, by acid catalyzed reaction with aldehydes.

U.S. Pat. Nos. 4,695,625 and 4,925,933 disclose the preparation of 16,17-acetals by trans-acetalization of the corresponding 16,17-acetonides.

PCT publication No. WO 02/38584 discloses a trans-acetalization method that yields a ciclesonide intermediate, which is readily converted into ciclesonide by subsequent esterification of the 21-alcohol.

According to the processes disclosed in the above patents, ciclesonide is obtained as a mixture of R and S epimers. All of these known processes lead to a product containing levels of the (22S)-epimer, which are unacceptably high for an API.

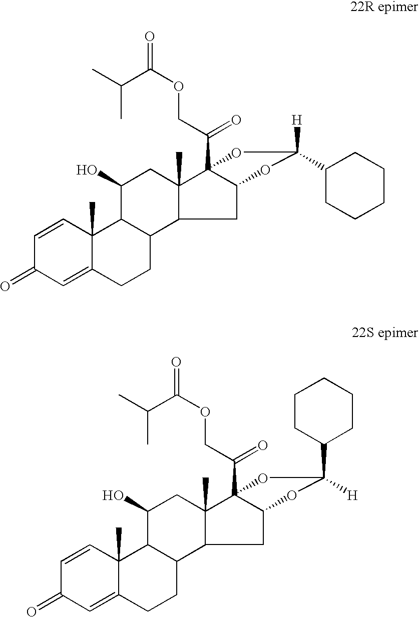

EP patent No. 929566 describes a process for the enrichment of the 22R-epimer of ciclesonide by fractional crystallization from a solution containing an R/S-epimer mixture in a mixture of water and a suitable water-miscible organic solvent.

There is a need in the art for additional ways to enrich ciclesonide.

### SUMMARY OF THE INVENTION

In one embodiment, the invention provides a process for increasing the 22R/22S epidemic ratio of ciclesonide comprising crystallizing ciclesonide from a solution of ciclesonide in at least one water-immiscible organic solvent.

In another embodiment, the present invention also provides a process for increasing the 22R/22S epidemic ratio of ciclesonide by crystallizing ciclesonide from a water immiscible organic solvent, and recycling the 22R epimer from the mother liquor of the crystallization processes.

In yet another embodiment, the present invention provides a process for enriching the 22R-epimer of ciclesonide comprising:

a) preparing a solution of ciclesonide having a 22S-epimer content of up to about 15% in a first anhydrous non-hydroxylic organic solvent or a mixture thereof with a second organic solvent having a lower-boiling than the first organic solvent, at a temperature between ambient temperature and reflux temperature of the solvent or solvent mixture;

b) crystallizing the 22R-epimer enriched ciclesonide.

Optionally, the crystallization step (b) may be repeated to further enrich the 22R-epimer content of ciclesonide.

### DETAILED DESCRIPTION OF THE INVENTION

The present invention provides a process for increasing the 22R/22S epidemic ratio of ciclesonide by using an anhydrous solvent system of water-immiscible organic solvents during crystallization. Not to be limited by theory, it is believed that anhydrous conditions allow for the effective use of water-immiscible organic solvents during crystallization and/or recrystallization. Typically, anhydrous conditions relate to a water content of less than 2% by weight, preferably, less than 1% by weight, more preferably, less than 0.5% by weight. This belief was confirmed when some water-immiscible organic solvents were found to possess good selectivity in separating the 22R-epimer of ciclesonide from its 22S-epimer, due to the increased solubility of the 22S epimer in these solvents. Additionally, anhydrous conditions reduce ciclesonide decomposition during the crystallization, particularly the tendency of the ester group of ciclesonide to be hydrolyzed in ethanol/water mixtures.

As used herein, unless otherwise defined, the term “ambient temperature” refers to a temperature of between about 20° C. to about 25° C.

The invention encompasses processes for increasing the 22R/22S epidemic ratio of ciclesonide by crystallizing cyclesonide from a water-immiscible organic solvent. This process comprises dissolving ciclesonide in at least one water-immiscible organic solvent to form a solution; crystallizing ciclesonide from the solution; and recovering the crystallized ciclesonide. After the first crystallization, a solid is obtained which can be crystallized again to further increase its epidemic purity. Preferably the water-immiscible organic solvent is a non-hydroxylic organic solvent.

The starting ciclesonide can be made using methods known in the art, such as the methods disclosed in U.S. Pat. Nos. 2,990,401; 3,929,768; 4,695,625, 4,925,933; 5,482,934; and 5,728,826, and disclosed in PCT publications WO 98/09982, hereby incorporated by reference. Preferably, the starting ciclesonide contains no more than about 15% of the 22S-epimer. More preferably, the starting ciclesonide contains no more than about 12% of the 22S-epimer.

Water-immiscible organic solvents include non-hydroxylic organic solvent. The non-hydroxylic organic solvents are organic solvents that lack a hydroxyl group in the chemical compound. Typically, the non-hydroxylic organic solvent includes, C₁-C₁₂ straight, branched or cyclic alkanes, C₂ to C₁₂ straight, branched or cyclic ethers. Preferably, the C₁-C₁₂ straight, branched or cyclic alkanes are C₆-C₁₂ straight, branched or cyclic alkanes, more preferably, C₆-C₈ straight, branched or cyclic alkanes. Preferably, the C₂ to C₁₂ straight, branched or cyclic ethers are C₅ to C₁₂ straight, branched or cyclic ethers, more preferably, C₅ to C₆ straight, branched or cyclic ethers. Specific examples of C₁-C₁₂ straight, branched or cyclic alkanes include heptane, hexane, and isooctane. Specific examples of C₂ to C₁₂ straight, branched or cyclic ethers include tert-butyl methyl ether, and diisopropyl ether. Preferably, the non-hydroxylic organic solvent is isooctane.

The crystallization process may further employ a second organic solvent, wherein the term second organic solvent relates to an organic solvent that has a boiling point lower than the non-hydroxylic organic solvent. The lower-boiling organic solvent can be any solvent that can dissolve the starting mixture of ciclesonide 22R/22S epimers. Preferably, the lower-boiling organic solvent includes C₁ to C₈ alcohols, C₂ to C₈ ketones, C₁₋₆ aliphatic halocarbons. Preferably, the C₁ to C₈ alcohols is C₁ to C₅ alcohols, more preferably, C₁ to C₄ alcohols. Preferably, the C₂ to C₈ ketones are C₂ to C₅ ketones, more preferably, C₂ to C₃ ketones. Preferably, the C₁₋₆ aliphatic halocarbons are C₁₋₄ aliphatic halocarbons, more preferably, C₁₋₂ aliphatic halocarbons. Specific examples of C₁ to C₈ alcohols include methanol, ethanol, and tert-butanol. Specific examples of C₂ to C₈ ketones include acetone. Specific examples of C₁₋₆ aliphatic halocarbons include dichloromethane. More preferably, the lower-boiling organic solvent is either acetone or dichloromethane, and most preferably, dichloromethane. The co-solvent is preferably technical grade, i.e. containing less than about 2% water by weight, preferably, less than 1% of water, more preferably, less than 0.5% by weight.

Typically, when a mixture of a water-immiscible organic solvent and a lower-boiling organic solvent is used, the ratio of the solvents is 20:1 by weight, respectively. Preferably, the ratio is 10:1, and more preferably, the ratio is 5:1 by weight. Optionally, the lower-boiling organic solvent may be removed by evaporation prior to inducing precipitation of the crystalline ciclesonide.

Preferably, the crystallization is performed by dissolving the starting ciclesonide in the water-immiscible organic solvent at a temperature ranging from ambient temperature to about the boiling point of the water-immiscible organic solvent to form a solution, concentrating the solution to obtain a suspension, and cooling the suspension to precipitation of solid ciclesonide. In the embodiment wherein a second lower-boiling organic solvent is used, the process preferably comprises dissolving the starting ciclesonide in the second lower-boiling organic solvent at a temperature ranging from ambient temperature to the boiling point of the second lower-boiling organic solvent, and adding the water-immiscible organic solvent. The mixture may then be concentrated to remove most or all of the second lower-boiling organic solvent, which removal typically results in a suspension.

Typically, the suspension obtained either with or without the second solvent, is cooled to a temperature of about 80° C. to about 10° C. Preferably, the concentrating step is performed by removing the water-immiscible organic solvent by distillation.

The precipitate may be separated or recovered using methods commonly known to the skilled artisan. For example, the crystalline ciclesonide may be recovered by filtration. Optionally, the recovered crystalline ciclesonide is washed and dried.

The present invention further provides a process for enriching the 22R-epimer of ciclesonide comprising:

a) preparing a solution of ciclesonide having a 22S-epimer content of up to about 15% in a first anhydrous non-hydroxylic organic solvent or a mixture thereof with a second organic solvent having a lower-boiling than the first organic solvent, at a temperature between ambient temperature and reflux temperature of the solvent or solvent mixture;

b) crystallizing the 22R-epimer enriched ciclesonide. Prior to crystallization, the solution obtained in step a) may be concentrated, in order to remove all, or most, of the lower-boiling organic solvent. Repetition of steps a) and b) may be performed in order to further increase the R/S epidemic ratio. The R/S epidemic ratio may be increased to at least about 99.9/0.1% after 2 or 3 such repetitions.

Optionally, the crystallization process may be repeated to increase the R/S epidemic ratio to a desired level. Preferably, the crystallization process is repeated to obtain a 22R/22S epidemic ratio of at least about 99.5:0.5, more preferably at least 99.75:0.25, and most preferably at least 99.9:0.1. Typically, the 22R/22S epidemic ratio may be increased to at least about 99.0/1.0 area by HPLC, after two repetitions. Preferably, the crystallization process is repeated to obtain a 22R/22S epidemic ratio of at least about 99.9/0.1 after four repetitions.

The filtration performed in the recovery step of the processes of the present invention provides a filtrate comprising of ciclesonide mixture of the 22R and 22S epimers. Typically, the ciclesonide mixture contains at least 15% of the 22S epimer of ciclesonide. An additional amount of the 22R epimer can be obtained from the said ciclesonide mixture by recycling it from the filtrate. The recycling process comprises recovering the said mixture of epimers of ciclesonide from the filtrate; converting the 22S epimer to 22R epimer to obtain a mixture of ciclesonide enriched with the 22R epimer, recovering the enriched ciclesonide. The said mixture of ciclesonide mixture of epimers may be recovered by concentrating at least one filtrate obtained from the crystallization process and cooling the concentrate to precipitate the said ciclesonide mixture of epimers. Optionally, the precipitated ciclesonide mixture may be filtered, rinsed, and dried.

Typically, the conversion of 22S epimer to 22R epimer is done by treating the precipitated ciclesonide mixture obtained from the filtrate with hydrofluoric acid, to obtain an enriched ciclesonide mixture; wherein the 22R epimer is enriched. The enriched ciclesonide mixture can then be isolated. The enriched ciclesonide may have the 22R epimer in an amount up to about 90-92%. The step of treating solid ciclesonide with hydrofluoric acid may optionally include the addition of a trace amount of cyclohexanecarboxaldehyde.

The enriched ciclesonide mixture may be further subjected to a crystallization process as described before.

The present invention also provides a process for increasing the 22R/22S epidemic ratio of ciclesonide by crystallization from a water immiscible solvent, and recycling the 22R epimer from the mother liquor of the crystallization processes.

Having described the invention with reference to certain preferred embodiments, other embodiments will become apparent to one skilled in the art from consideration of the specification. The invention is further defined by reference to the following examples describing in detail the process of the invention. It will be apparent to those skilled in the art that many modifications, both to materials and methods, may be practiced without departing from the scope of the invention.

### EXAMPLES

The HPLC analysis was carried out using the following equipment and methodology. The column was a Prodigy ODS, 220×4.6 mm, 5 μm. The eluent was ethanol/water 50/50 at a flow rate of 2 mL/min. The detector was an UV-DAD at 242 nm.

### Example 1a

Preparation of Ciclesonide (Having an 22R/22S-Epimer Ratio of 90:10)

Desonide 21-isobutyrate (70 g, 144 mmol) was added in portions at a temperature of about −20° C. to hydrofluoric acid (73%, 350 g), and to the resulting solution was added cyclohexanecarboxaldehyde (18.4 g, 164 mmol) over ca. 5 minutes. The reaction mixture was held at −10° C. to −15° C. for 1 hour, then at ca. −30° C. for 2 hours, and then poured into an ice-cold mixture of ammonium hydroxide solution (26% 87.5 g) and water (2625 g). After stirring the suspension for 1 hour, a precipitate appeared which was collected and rinsed with water. In order to ensure the absence of acidity, the humid precipitate was distributed between dichloromethane (1000 g) and water (1000 g, adjusted to pH 8 with ammonium hydroxide solution). The organic phase was concentrated at atmospheric pressure to an oily residue (crude product) having a 22R/22S epidemic ratio of about 90/10 as determined by HPLC.

### Example 1b

Enrichment Process—First Crystallization

The oily residue of Example 1a (theoretical yield: 77.8 g) was dissolved in acetone (280 g) heated at reflux and the solution was diluted, whilst maintaining under reflux, with isooctane (1400 g) and concentrated at atmospheric pressure until the temperature of the suspension reached 90° C. The suspension was cooled under agitation at about 70° C. during 30 minutes, and the crystalline precipitate was collected by filtration and rinsed with isooctane. The crystals were dried at 80° C. under vacuum to give 64 grams of ciclesonide with an R/S epimer ratio 96.5/3.5 as determined by HPLC.

### Example 1c

Enrichment Process—Second Crystallization

The product of Example 1b was recrystallized in the same manner as disclosed in Example 1b using acetone (96 g) and isooctane (1400 g) to give 56.8 grams of ciclesonide with an R/S epimer ratio 98.3/1.7 as determined by HPLC.

### Example 1d

Enrichment Process—Third Crystallization

The product of Example 1c was recrystallized in the same manner as disclosed in Example 1b using acetone (85 g) and isooctane (1400 g) to give 50.5 grams of ciclesonide with an R/S epimer ratio 99.3/0.7 as determined by HPLC.

### Example 1e

Enrichment Process—Fourth Crystallization

The product of Example 1 d was recrystallized in the same manner as disclosed in Example 1b using acetone (76 g) and isooctane (1400 g) to give 45.9 grams of ciclesonide with an R/S epimer ratio 99.75/0.25.

### Example 2

Conversion of Epimers—Second-Crop Recycling

The combined mother liquors of the crystallization processes in Examples 1b-1e were concentrated to a volume of about 400 mL and cooled to about 10° C. The precipitate was collected by filtration, rinsed with isooctane (40 g), and dried at 80° C. under vacuum to give 20 grams of ciclesonide with an R/S epimer ratio 80/20. This second-crop material could be re-equilibrated into ciclesonide having the equilibrium R/S epimer ratio of 92/8 by treatment with 73% hydrofluoric acid according to Example 1.

### Example 3

Enrichment Process—Ciclesonide Having a Ratio of 99.9/0.1

Ciclesonide (27.8 g, epimer ratio 99.76/0.24) was dissolved in dichloromethane (220 g) under reflux, and the solution was diluted with isooctane (880 g) and concentrated at atmospheric pressure until the temperature of the resulting suspension reached 90° C. (complete removal of dichloromethane). The suspension was allowed to cool under agitation to about 70° C. during 30 minutes, and the precipitate was collected by filtration and rinsed with isooctane. The crystals were dried at 80° C. under vacuum to give 24.8 grams of ciclesonide with an R/S epimer ratio 99.9/0.1.

### Example 4

Preparation of Ciclesonide (Having an 22R/22S-Epimer Ratio of 90:10)

A mixture of desonide (65.4 g), acetone (524 g), isobutyric anhydride (37.3 g) and potassium carbonate anhydrous (41.8 g) was heated under reflux for 90 minutes, then cooled to about 40° C. and diluted with water (131 g). The solution was concentrated until 260 grams of solvent had evaporated, cooled, and poured into water (1635 g) under agitation at about 5° C. to obtain a precipitate. The precipitate was collected by filtration, rinsed with water, and dried at 80° C. under vacuum to give 76.2 grams (99.7% of theory) of desonide 21-isobutyrate.

Desonide 21-isobutyrate (70 g, 144 mmol) was added in portions at about −20° C. to 73% hydrofluoric acid (350 grams). To the resulting solution was added cyclohexanecarboxaldehyde (18.2 g) over about 5 minutes, the mixture was stirred at about −10° C. for 2 hours, and then poured into an ice-cold mixture of 26% ammonium hydroxide solution (875 g) and water (2625 g). The suspension was stirred for 1 hour, and the precipitate was collected by filtration and rinsed with water.

In order to ensure the absence of acidity, the humid precipitate was distributed between dichloromethane (350 g) and water (1000 g, adjusted to pH 8 with ammonium hydroxide solution). The organic phase was concentrated at atmospheric pressure to an oily residue. The residue was dissolved in acetone (210 g) and the solution was poured into water (2100 g) under agitation. The precipitate was collected by filtration, rinsed with water, and dried at 80° C. under vacuum to give 77.5 g (99.6% of theory) of ciclesonide with an R/S epimer ratio of about 90/10.

### Example 5a

Enrichment Process—First Crystallization

The starting material was crude ciclesonide prepared according to Example 1 or Example 4 of EP patent No. 929566 (page 4 lines 11-22). Crude ciclesonide (60 g, epimer ratio 90/10) was dissolved in dichloromethane (300 g) and diluted with isooctane (1200 g), thereafter, the solution was concentrated at atmospheric pressure until the temperature reached 90° C. (complete removal of dichloromethane). The suspension was allowed to cool under agitation to about 70° C. during 30 minutes, the precipitate was collected by filtration, and rinsed with isooctane. The crystals were dried at 80° C. under vacuum to give 51.5 grams of ciclesonide with an R/S epimer ratio 94.4/5.6. The mother liquors had an R/S ratio of ca. 56/44, which demonstrates the excellent selectivity of the process for removal of the undesired epimer.

### Example 5b

Enrichment Process—Second Crystallization

The product of Example 5a was recrystallized in the same manner as described in Example 5a using the same quantities of dichloromethane and isooctane to give 44 grams of ciclesonide with an R/S epimer ratio 97.5/2.5. Note: Examples 5b to 5d start with the amount obtained in the preceding example.

### Example 5c

Enrichment Process—Third Crystallization

The product of Example 5b was recrystallized in the same manner as described in Example 5a using the same quantities of dichloromethane and isooctane to give 40 grams of ciclesonide with an R/S epimer ratio 98.7/1.3.

### Example 5d

Enrichment Process—Fourth Crystallization

The product of Example 5c was recrystallized in the same manner as described in Example 5a using the same quantities of dichloromethane and isooctane to give 37 grams of ciclesonide with an R/S epimer ratio 99.5/0.5. This represents a yield of about 62% without extracting the epimer from the filtrates or mother liquors.

### Example 6a

Second-Crop Recycling

The combined mother liquors of the crystallization processes in Examples 5a-5d were concentrated at atmospheric pressure to obtain a suspension that was cooled to ambient temperature. The precipitate was collected by filtration and rinsed with isooctane. The crystals were dried at 80° C. under vacuum to give 20 g of ciclesonide with an R/S epimer ratio 73/27.

### Example 6b

Recycling by Re-Equilibration of Second Crop

The second-crop material (20 grams) obtained in Example 6a was dissolved in 73% hydrofluoric acid (100 g) and the solution stirred at −30° C. for 2 hours, then isolated as described in Example 4. 19.5 grams of ciclesonide having an R/S ratio of ca. 90/10 were obtained (yield: 89% w/w).

### Example 7a

Enrichment Process—First Crystallization

Crude ciclesonide (2 g, epimer ratio 90/10, prepared as in Example 4) were dissolved in isooctane (1000 g) and allowed to cool under agitation to ambient temperature. The precipitate was collected by filtration, rinsed with isooctane, and dried at 80° C. under vacuum to give 1.4 grams of ciclesonide with an R/S epimer ratio 98.5/1.5.

### Example 7b

Enrichment Process—Second Crystallization

The product of Example 7a was recrystallized in the same manner as described in Example 7a using isooctane (700 g) to give 1.1 g of ciclesonide with an R/S epimer ratio 99.6/0.4.

### Example 8

Preparation of Ciclesonide (Having an 22R/22S-Epimer Ratio of 92:8)

16α-hydroxyprednisolone 21 isobutyrate (25 g) was added in portions to hydrofluoric acid (125 g, 73%) at ca. −20° C. To the resulting solution was added, during ca. 5 minutes, cyclohexanecarboxaldehyde (6.6 g) and the mixture was stirred at ca. −10° C. for 1 hour, then at −20° C. to −30° C. for 1.5 hours, and poured into an ice-cold mixture of 26% ammonium hydroxide solution (312 g) and water (940 g). The suspension was stirred for 1 hour, the precipitate was collected by filtration, and rinsed with water. In order to ensure the absence of acidity, the humid precipitate was distributed between dichloromethane (250 g) and water (125 g, adjusted to pH 8 with ammonium hydroxide solution). The organic phase was concentrated at atmospheric pressure to an oily residue. The oily residue was dissolved in acetone (75 g) and the solution was poured into water (750 g, at ca. 0° C.) under agitation. The precipitate was collected by filtration, rinsed with water, and dried at 80° C. under vacuum to give 29 grams of ciclesonide with an R:S epimer ratio of ca. 92:8.

### Example 9

Decomposition in Ethanol-Water Mixture

20 mg ciclesonide were dissolved in 6 mL ethanol and 4 mL purified water and the solution was kept at 80° C. for 64 hours. The initial purity of 99.74% decreased over this period to 95.34% with formation of 3.99% of the corresponding 21-hydroxy analogue

What is claimed is:

1. A process for increasing the 22R/22S epidemic ratio of ciclesonide comprising crystallizing ciclesonide from a solution of ciclesonide in at least one water immiscible organic solvent.

2. A process according to claim 1 comprising:
a) dissolving ciclesonide in at least one water immiscible organic solvent to form a solution;
b) crystallizing ciclesonide from the solution; and
c) isolating the crystallized ciclesonide.

3. The process according to claim 1, wherein the starting ciclesonide has no more than about 15% of the 22S-epimer of ciclesonide.

4. The process according to claim 3, wherein the starting ciclesonide contains no more than about 12% of the 22S-epimer of ciclesonide.

5. The process according to claim 1, wherein the water immiscible organic solvent is a non-hydroxylic organic solvent.

6. The process of claim 1, wherein the water immiscible organic solvent is selected from the group consisting of C₁-C₁₂ straight, branched or cyclic alkanes, and C₂ to C₁₂ straight, branched or cyclic ethers.

7. The process of claim 6, wherein the solvent is a C₆-C₈ straight, branched or cyclic alkanes.

8. The process of claim 6, wherein the solvent is a C₅ to C₁₂ straight, branched or cyclic ether.

9. The process of claim 6, wherein the solvent is a C₅ to C₆ straight, branched or cyclic ether.

10. The process according to claim 1, wherein the water immiscible organic solvent is heptane, hexane, isooctane, tert-butyl methyl ether, or diisopropyl ether.

11. The process according to claim 1, wherein the water immiscible organic solvent is isooctane.

12. The process according to claim 1, wherein the solution further includes at least one organic solvent having lower boiling point than said water immiscible organic solvent.

13. The process of claim 12, wherein the lower boiling point solvent is selected from the group consisting of C₁ to C₈ alcohols, C₂ to C₈ ketones and C_{1 to C}₆ aliphatic halocarbons.

14. The process of claim 12, wherein the solvent is a C₁ to C₅ alcohol.

15. The process of claim 12, wherein the solvent is a C₁ to C₄ alcohol.

16. The process of claim 12, wherein the ketone is a C₂ to C₅ ketone.

17. The process of claim 12, wherein the ketone is a C₂ to C₃ ketone.

18. The process of claim 12, wherein the halocarbon is a C₁₋₄ aliphatic halocarbon.

19. The process of claim 12, wherein the halocarbon is a C₁₋₂ aliphatic halocarbon.

20. The process according to claim 12, wherein the lower-boiling organic solvent is dichloromethane, acetone, methanol, ethanol, or tert-butanol.

21. The process according to claim 12, wherein the lower-boiling organic solvent is acetone or dichloromethane.

22. The process according to claim 12, wherein the lower-boiling organic solvent is dichloromethane.

23. The process according to claim 12, wherein the ratio of water immiscible organic solvent to lower-boiling organic solvent is 20:1 by weight, respectively.

24. The process according to claim 23, wherein the ratio of water immiscible organic solvent to lower-boiling organic solvent is about 10:1 weight, respectively.

25. The process according to claim 24, wherein the ratio of water immiscible organic solvent to lower-boiling organic solvent is about 5:1 by weight, respectively.

26. The process according to claim 12, further comprising removing the lower-boiling organic solvent prior to crystallizing the ciclesonide.

27. The process according to claim 1, wherein crystallizing ciclesonide comprises concentrating the solution to obtain a suspension, and cooling the suspension to induce precipitation of crystalline ciclesonide.

28. The process according to claim 27, wherein the suspension is cooled to a temperature of about 80° C. to about 10° C.

29. The process according to claim 1, wherein the process is repeated and the crystallized ciclesonide is used as the starting ciclesonide.

30. The process according to claim 1, wherein the 22R/22S epidemic ratio is increased to at least about 99.0/1.0 after repeating the process twice.

31. The process according to claim 1, wherein the 22R/22S epidemic ratio is at least about 99.9/0.1 after repeating the process four times.

32. A process according to claim 1, further comprising recycling the 22R epimer from the filtrate obtained in step c.

33. The process according to claim 32, wherein the recycling comprises concentrating at least one filtrate having an epidemic mixture of 22S and 22R of ciclesonide, converting the 22S epimer to 22R epimer, and isolating ciclesonide having an increased 22R/22S epidemic ratio as compared to the ciclesonide of the filtrate.

34. A process for enriching the 22R-epimer of ciclesonide comprising:
a) preparing a solution of ciclesonide having a 22S-epimer content of up to about 15% in a first anhydrous non-hydroxylic organic solvent or a mixture thereof with a second organic solvent having a lower-boiling than the first organic solvent, at a temperature between ambient temperature and reflux temperature of the solvent or solvent mixture;
b) crystallizing the 22R-epimer enriched ciclesonide.

35. The process of claim 34, wherein the crystallization step (b) is repeated to further enrich the 22R-epimer content of ciclesonide.
